(19) United States
(12) Patent Application Publication
(10) Pub. No.: US 2016/0121074 A1
(43) Pub. Date: May 5, 2016

(54) System with a Heart Rate Adjusting Mechanism

(71) Applicant: ICON Health & Fitness, Inc., Logan, UT (US)

(72) Inventor: Darren C. Ashby, Richmond, UT (US)

(21) Appl. No.: 14/932,455

(22) Filed: Nov. 4, 2015

Related U.S. Application Data
(60) Provisional application No. 62/075,744, filed on Nov. 5, 2014.

(51) Int. Cl. A61M 21/02; A61B 5/00; A61B 5/024

(52) U.S. Cl. CPC A61M 21/02; A61B 5/024; A61B 5/6891; A61B 5/4815; A61M 2021/0022

(57) ABSTRACT

A system for adjusting a heart rate includes an interface for communicating with a heart rate monitor and a speaker. The system also includes a processor and memory. The memory includes programmed instructions to cause the processor to determine a natural heart rate of a user, determine a target rate for the user, and cause a sound to be emitted from the speaker to adjust the natural heart rate to the target heart rate.

### RELATED APPLICATIONS

This application claims priority to U.S. Patent Application Ser. No. 62/075,744 titled “System with a Heart Rate Adjusting Mechanism” and filed on 5 Nov. 2014, which application is herein incorporated by reference for all that it discloses.

### BACKGROUND

Sleep provides many benefits to humans and animals. While there is still much to learn about the process and benefits of sleep, research suggests that during sleep, restorative functions occur in the nervous, skeletal, and muscular systems. Also, memory loss has been associated with sleep deprivation suggesting that sleep plays a role in retaining memory. Some experts believe that people should get at least six hours of sleep a night. However, due to sleep disorders, busy schedules, inconvenient environments, and life choices, some find getting adequate quality sleep difficult.

U.S. Pat. No. 5,479,939 issued to Hiroyuki Ogino describes one type of device for determining whether a person is asleep in order to further evaluate the sleeping subject. In this reference, movement of a person in bed is detected without contacting the body, and time measurement is reset and started newly by a timer every time a detected movement exceeds a predetermined set value. When the measurement time of the timer exceeds a set time predetermined, it is judged that the body has fallen asleep on the bed. Meanwhile, absence or presence in bed and rough body movement are judged by detecting the fine body movement propagated by the functioning of the heart and the breathing of the body. Another type of system for using sensors to detect the sleeping conditions of a patient is described in U.S. Patent Publication No. 2009/0178199 issued to Andreas Brauers, et al. Each of these documents are herein incorporated by reference for all that they contain.

### SUMMARY

In one aspect of the invention, a system for adjusting a heart rate includes an interface for communicating with a heart rate monitor and a speaker.

In one aspect of the invention, the system includes a processor and memory.

In one aspect of the invention, the memory includes programmed instructions to cause the processor to determine a natural heart rate of a user.

In one aspect of the invention, the memory includes programmed instructions to cause the processor to determine a target heart rate for the user.

In one aspect of the invention, the memory includes programmed instructions to cause the processor to generate a sound to be emitted from the speaker to adjust the natural heart rate to the target heart rate for the user.

In one aspect of the invention, the heart rate monitor is in communication with the communications interface.

In one aspect of the invention, the speaker is in communication with the interface.

In one aspect of the invention, the target heart rate is configured to assist the user with sleeping.

In one aspect of the invention, the sound is adjusted over a time period.

In one aspect of the invention, the sound at a beginning of the time period approximates the natural heart rate and the sound at an end of the time period approximates the target heart rate.

In one aspect of the invention, the sound is changed incrementally during the time period.

In one aspect of the invention, the heart rate monitor is incorporated into a bed.

In one aspect of the invention, the speaker is incorporated into the bed.

In one aspect of the invention, the sound is propagated through a medium of the bed.

In one aspect of the invention, the heart rate monitor is in wireless communication with the processor.

In one aspect of the invention, the target heart rate is determined based on a percentage reduction of a resting heart rate of the user.

In one aspect of the invention, a system for adjusting a heart rate includes a communication interface.

In one aspect of the invention, the system includes a heart monitor in communication with the communication interface.

In one aspect of the invention, the system includes a speaker in communication with the communication interface.

In one aspect of the invention, the system includes a processor and memory.

In one aspect of the invention, the memory comprises programmed instructions executable by the processor to determine a natural heart rate of a user.

In one aspect of the invention, the memory comprises programmed instructions executable by the processor to determine a target heart rate configured to assist a user with sleeping.

In one aspect of the invention, the memory comprises programmed instructions executable by the processor to cause a sound to be emitted from the speaker to adjust the natural heart rate to the target heart rate where the sound is adjusted over a time period such that the sound at a beginning of the time period approximates the natural heart rate and the sound at an end of the time period approximates the target heart rate.

In one aspect of the invention, the sound is changed incrementally during the time period.

In one aspect of the invention, the heart rate monitor is incorporated into a bed.

In one aspect of the invention, the speaker is incorporated into the bed.

In one aspect of the invention, the sound is propagated through a medium of the bed.

In one aspect of the invention, the heart rate monitor is in wireless communication with the processor.

In one aspect of the invention, the target heart rate is determined based on a percentage reduction of a resting heart rate of the user.

In one aspect of the invention, a system for adjusting a heart rate includes a communication interface.

In one aspect of the invention, the system includes a heart rate monitor incorporated into a bed and in communication with the communication interface.

In one aspect of the invention, the system includes a speaker incorporated into a bed and in communication with the communication interface.

In one aspect of the invention, the system includes a processor and memory.

In one aspect of the invention, the memory includes programmed instructions executable by the processor to determine a natural heart rate of a user.

In one aspect of the invention, the memory includes programmed instructions executable by the processor to determine a target heart rate configured to assist a user with sleeping.

In one aspect of the invention, the memory includes programmed instructions executable by the processor to cause a sound to be emitted from the speaker to adjust the natural heart rate to the target heart rate where the sound is adjusted over a time period such that the sound at a beginning of the time period approximates the natural heart rate and the sound at an end of the time period approximates the target heart rate.

Any of the aspects of the invention detailed above may be combined with any other aspect of the invention detailed herein.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a perspective view of an example system incorporated into a bed in accordance with the present disclosure.

FIG. 2 illustrates a top view of a system including a heart rate monitor incorporated into a bed in accordance with the present disclosure.

FIG. 3 is a diagram illustrating a natural heart approximating a generated heart rate in accordance with the present disclosure.

FIG. 4 is a block diagram illustrating a heart rate adjustment system in accordance with the present disclosure.

FIG. 5 is a block diagram illustrating a method for adjusting a natural heart rate in accordance with the present disclosure.

Throughout the drawings, identical reference numbers designate similar, but not necessarily identical, elements.

### DETAILED DESCRIPTION

A heart rate adjustment system including a heart rate mimicking system is disclosed herein. Specifically, the present system provides a system for determining a user's current heart rate when attempting to sleep, identifying a target heart rate for efficient sleep, and generating a simulated heart rate configured to gradually encourage the user's heart rate to trend toward the target heart rate for efficient sleep.

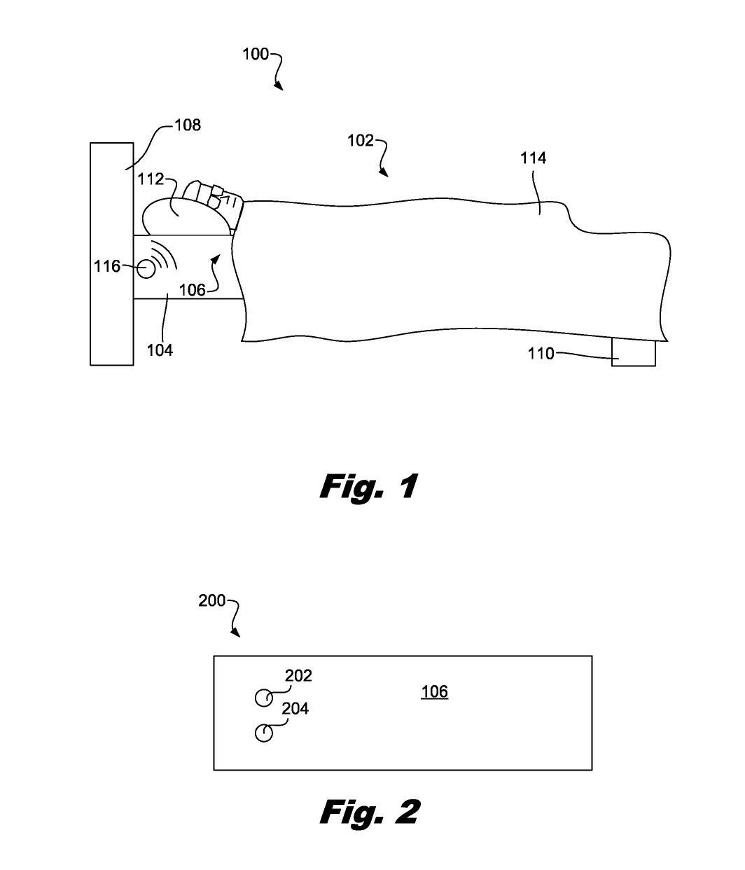

With reference to the figures, FIG. 1 illustrates a perspective view of an example heart rate adjustment system 100 incorporated into a bed 102. In the illustrated example, the bed 102 includes a bed frame 104, a mattress 106, a head board 108, bed legs 110, a pillow 112, and a blanket 114. When a user desires to sleep, the user may lay down on the mattress 106 and rest his or her head on the pillow 112. Depending on the temperature in the room, the user may desire to pull the blanket 114 over himself or herself.

When a healthy user lies down to sleep, the user may drift into the initial stages of sleep which are characterized by the user being in a semi-conscious state. As time progresses, the user typically progresses into a deeper sleep. The first stages of sleep experienced by the user are referred to as non-rapid eye movement (non-REM) sleep. Often, during non-REM sleep, the user's body advances into a condition where the user's heart rate slows down, the user's breathing gets deeper and slower, and the user's muscles become more relaxed.

The final stage of sleep is rapid eye movement (REM) sleep where the user's brain activity and heart rate pick up again. During REM sleep, the user's eyes move side to side and the user may experience dreaming. REM sleep is the deepest sleep and generally, the user's muscles are often inhibited from moving during this stage of sleep. It may take a user between 90 and 120 minutes to advance through a single cycle of sleep. Upon completion of the first cycle, the user generally advances through the stages of the sleep cycle again. Often a user may complete four to six sleep cycles in a given night.

Some users may experience sleep disorders where it is difficult for the user to initially fall asleep or to stay asleep. To assist such users, the adjustment system 100 may detect the user's heart beat with a heart rate monitor. Any appropriate type of heart rate monitor may be used in accordance with the principles described in the present disclosure. For example, the user may wear a heart rate monitor that is in communication with a processor of the adjustment system 100. In such an example, the heart rate monitor may be a chest strap monitor, a wrist watch monitor, a monitor worn by the user, a monitor incorporated into the user's clothing, another type of heart rate monitor, or combinations thereof. Further, according to one embodiment, the heart rate monitor may detect electrical signals that are produced during the operation of a beating heart. Such electrical signals may be recorded by at least two electrodes in contact with the user's skin. However, other mechanisms for determining the user's heart rate may be used. For example, a microphone may be placed within a region where the microphone can pick up on the sounds made by the user's heartbeat. Further, the heart rate monitor may include a mechanism for detecting the user's pulse, and the heart rate monitor may determine the user's heart rate based on the pulse rate. While these examples have been described with reference to specific heart rate monitors, any appropriate mechanism for determining the user's heart rate may be used.

In addition to determining the user's heart rate, the adjustment system 100 may also determine a target heart rate that may assist the user with sleeping, either initially falling asleep, or maintaining an appropriate level of sleep. For example, the target heart rate, may be a heart rate that is associated with the user when the user enters into the early stages of sleeping. For example, non-REM stages of the sleep cycle are often characterized by a heart rate drop, and the target heart rate may be a heart rate exhibited by the user during such non-REM stages. In some cases, a user's target heart rate is about 6.0 to 10.0 percent lower than the user's resting heart rate. However, target heart rates may be affected by a host of factors including the user's age, weight, body composition, gender, overall fitness level, diet, other health factors, other factors, or combinations thereof. According to one embodiment, the target heart rate is mathematically calculated based on average heart rate drops. Alternatively, the user's heartbeat may be tracked during a sleep cycle, and the various heart rates associated with each sleep stage may be recorded and used as target heart rate in future iterations.

Once the target heart rate is determined, the adjustment system 100 may cause a sound that mimics the target heart rate to be directed towards the user. In some examples, a speaker 116 is incorporated into the bed 102, and the processor can cause the sounds to be emitted from the speaker 116. In such an example, the sounds can have the effect of causing the user's heart rate to slow down to the same level as the target heart rate. By bringing down the user's heart rate, the user may start to advance into the early stages of the sleeping cycle. Thus, the sounds may assist the user with falling asleep.

In the illustrated example, the speaker 116 is incorporated into the bed frame 104. In such an example, the sounds may be directed to the user through the air and through the bed frame 104 or other materials that make up the bed. In some examples, the speaker 116 emits the sounds such that the sounds are transmitted through the air and the user picks up the sounds primarily through his or his auditory system. While the present exemplary system is described, for simplicity, in the context of a speaker system providing an audible heartbeat, in other examples, the sounds are directed to the user primarily through vibrations in the media of the bed, and the user primarily picks up the sounds through his or her tactile senses. According to this exemplary embodiment, a transducer is associated with or incorporated into the bed. The transducer receives a signal from the adjustment system 100 and converts the signal into vibrations. However, any appropriate mechanism for directing the sound to the user may be used in accordance with the principles described herein.

While the examples described above make specific reference to the sounds or vibrations mimicking the target heart rate, the sounds may produce a beat that is slower than the user's natural heart rate, but faster than the target heart rate. Such intermediary heart rate sounds may be used to slowly adjust the user's heart rate to the desired target heart rate. For example, the sounds may produce a beat that is 5.0 percent slower than the user's natural heart rate for a predetermined increment of time. During that increment of time, the user's heart rate will tend to mimic the produced beat, slowing down to have the same rate as the beat of the sounds. At the conclusion of the incremental time period, another slower beat may be caused to be emitted from the speaker 116. As before, the user's heart rate may also slow down to mimic the rate of the subsequent sound. This process may repeat itself until the user's heart rate arrives at the target heart rate, where the target heart rate continues to be generated by the speaker 116 or transducer.

The increments of time may be any appropriate length. For example, the time increments between additional adjustments may be for 10.0 seconds, 20.0 seconds, 30.0 seconds, 1.0 minute, 2.0 minutes, another duration, or combinations thereof. Alternatively, the system may dynamically detect the user's heart rate and only adjust the generated heart rate after a user's heart beat has appropriately mimicked the generated heart rate. Additionally, the increments of time may have different time lengths, which may depend on how much of a difference there is in the slower heart rate than the current heart rate of the user.

In some cases, the sounds are emitted through the speaker 116 just long enough for the user to establish a deep stage of sleep, wherein the user is left to naturally rest. In other cases, the sounds are emitted through just certain stages of sleep. For example, the sounds may be directed to the user during just the non-REM stages of sleep or just certain stages of the non-REM sleep. Since the user's heart rate varies and often increases during REM sleep, the sounds may be turned off during REM sleep to avoid influencing the user's heart rate during REM sleep. In yet other cases, the sounds are emitted though all of the sleep cycle's stages, including during REM sleep. In other examples, the sounds are just played at the conclusion of the user's REM sleep to assist the user in reentering the sleep cycle.

To determine the user's heart rate, the adjustment system 100 may have access to profile information about the user, such as the user's weight, body composition, height, age, gender, health conditions, other factors, or combinations thereof. Such profile information may be available to the adjustment system 100 through an online system such as the iFit program available through www.ifit.com and administered through ICON Health and Fitness, Inc. located in Logan, Utah, U.S.A. An example of a program that may be compatible with the principles described in this disclosure is described in U.S. Pat. No. 7,980,996 issued to Paul Hickman. U.S. Pat. No. 7,980,996 is herein incorporated by reference for all that it discloses. However, such profile information may be available through other types of programs that contain such information. For example, such information may be gleaned from social media websites, blogs, government databases, private databases, other sources, or combinations thereof. Also, the adjustment system 100 may record the user's heart rate through the night and send that information to the user's profile. Such information may allow the user to determine patterns about his or her sleep, become aware of sleeping conditions, track sleeping trends, establish baseline heart rates throughout the sleep cycle, perform other tasks, or combinations thereof. Further, the recorded information may be used by the adjustment system 100 to learn which target heart rates were the most effective for helping the user sleep. For example, if the calculated target heart rate appears to be less effective than another heart rate, the system may adapt to the other heart rate. In such examples, the target heart rates may be customized for each individual.

The adjustment system 100 may automatically turn on in response to detecting a heartbeat through the heart rate monitor. In other examples, the adjustment system 100 automatically activates in response to detecting a heart rate when the lights are out in the room with the bed 102. In yet other examples, the adjustment system 100 automatically activates in response to detecting a heart rate during certain time periods, such as the night time or evening. In other examples, the detecting of a heart rate is not used to activate the adjustment system. In such cases, the time of day, identification of the user through a camera, detection of a person on the bed through weight sensors, other mechanisms for detecting that conditions are right to activate the adjustment system 100, or combinations thereof may be used to active the adjustment system 100.

In yet other cases, the adjustment system 100 may activate in response to the user providing a command to the system to activate. For example, as the user lies down to sleep, the user may instruct the adjustment system 100 to turn on by flipping a switch, pressing a button, touching a touch screen input, sending a message through a mobile device, providing a speech command, providing instruction through another type of input mechanism, or combinations thereof.

In examples where the user wears his or her own heart rate monitor, the adjustment system 100 may determine the identity of the user based on an identifier of the heart rate monitor. In one example, the personal heart rate monitor may include an identification code in a signal that contains the heart rate information sent to the processor. In other examples, a camera may be located in the room with the bed, and the adjustment system 100 may identify the user through the camera.

FIG. 2 illustrates a top view of an example heart rate monitor 200 incorporated into a bed 102 in accordance with the present disclosure. In this example, a first electrode 202 and a second electrode 204 are incorporated into a mattress 106 of the bed 102. These electrodes 202, 204 may be used to detect a voltage that represents the user's heart rate. As the user lies down, the electrodes 202, 204 may come into contact with the user's skin such that the electrodes 202, 204 can detect electrocardiography (ECG) signals of the user.

The electrodes 202, 204 may be metal pieces that come into contact with any appropriate parts of the user's body when the user lies down on the mattress 106. In some examples, the electrodes 202, 204 are positioned to come into contact with the user's arms, chest, legs, neck, feet, wrists, upper body, lower body, other portions of the user's body, or combinations thereof.

In other examples, the electrodes 202, 204 come into contact with the user's skin indirectly. In such examples, the electrodes 202, 204 may be buried beneath the surface of the mattress 106, but the electrodes 202, 204 come into direct contact with an electrically conductive portion of the surface of the mattress 106. Such electrically conductive portions of the mattress 106 may be flexible to provide the user with more comfort as he or she lies down on the mattress 106. In some examples, the sheets on the mattress 106 and/or the user's clothing have electrically conductive portions of fabric that come into direct contact with either the electrodes 202, 204 or electrically conductive portions of the mattress 106. Thus, while the electrodes 202, 204 may not come into direct contact with the user's skin, an electrically conductive pathway may be formed between the electrodes 202, 204 and the user's skin such that the electrodes 202, 204 can detect the user's heart rate.

In other examples, the principles described above in relation to the electrodes 202, 204 incorporated into the mattress 106 may be applied to electrodes incorporated into other portions of the bed. For example, the electrodes 202, 204 may be incorporated into the bed frame 104, the pillow 112, the blanket 114, another portion of the bed 102, a watch, a bracelet, a chest-strap, a finger sensor, or combinations thereof.

While the examples above have been described with reference to heart rate monitors that use electrical contact to determine the user's heart rate, other types of heart rate monitors may be used in accordance with the principles described herein. For example, the inductive and capacitive mechanisms for determining the user's heart rate may be used in accordance with the principles described herein.

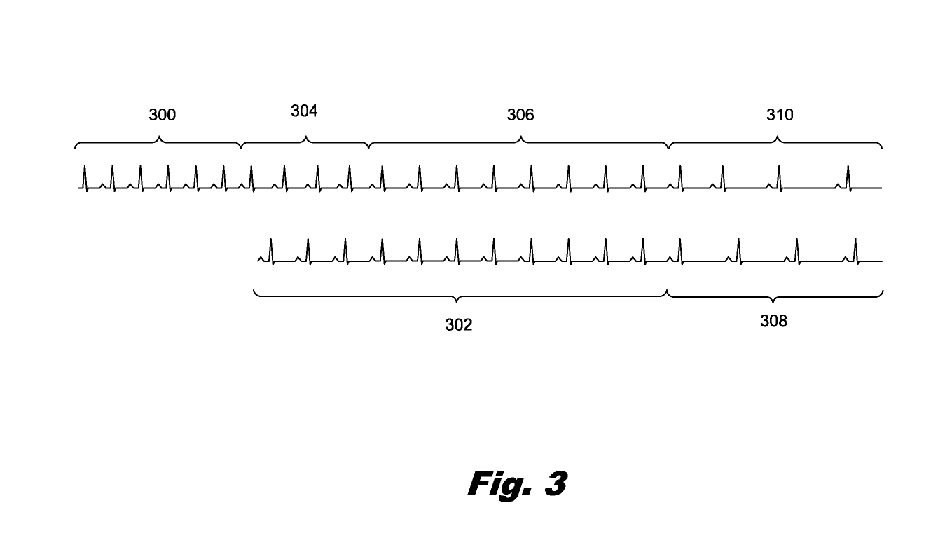

FIG. 3 illustrates a diagram of an example of changing a natural heart rate 300 in accordance with the present disclosure. In this example, the natural heart rate 300 is depicted as having a specific rate. As time passes, a first sound 302 or vibration is directed towards the user. The first sound has a slower beat than the natural heart rate 300. As the user perceives the first sound 302, the user's body causes the user's heart rate to mimic the beat of the first sound 302. Thus, the user's heart rate changes during a first transition phase 304. At the end of the first transition time 304, the user's current heart rate 306 has the same rate as the first sound 302.

In the example of FIG. 3, the adjustment system 100 changes the user's natural heart rate to the target heart rate through multiple incremental sounds with progressively slower beats. In the illustrated example, a second sound 308 or vibration is directed towards the user after the user's current heart rate 306 mimics the first sound 302 or vibration. The second sound 308 or vibration may be closer to the target heart rate than the first sound 302. As a result, the user's current heart rate 306 enters into a second transition phase 310. During the second transition phase 310, the user's current heart rate 306 slows down to mimic the beat rate of the second sound 308 or vibration.

This incremental process may repeat itself until the user's heart rate mimics the target heart rate with each of the sounds or vibrations directed towards the user during each time increment. During each time increment, the sounds may have beat rates that progressively get closer to the target heart rate.

The adjustment system 100 may have a single target heart rate at which the adjustment system 100 desires to impose on the user's heart rate. In such example, the user's heart rate may be brought to that rate, and the sound may cause the user's heart to maintain that rate. However, in other examples, the user's target heart rate may change over time. For example, the user's target heart rate may change depending on the stage of the user's sleep cycle. In some cases, the adjustment system 100 may determine that the target heart rate for the user during the second stage of sleep is to be different than the target heart rate during the user's third stage of sleep. Further, the same stage in a sleep cycle may have different preferred heart rate depending on the number of sleep cycles that the user has already gone through that night. For example, during the initial sleep stages of the first cycle, the adjustment system 100 may determine that the target heart rate is to have a first rate, while the target heart rate of the initial stages during the second sleep cycle is to have a second rate that is different than the first rate.

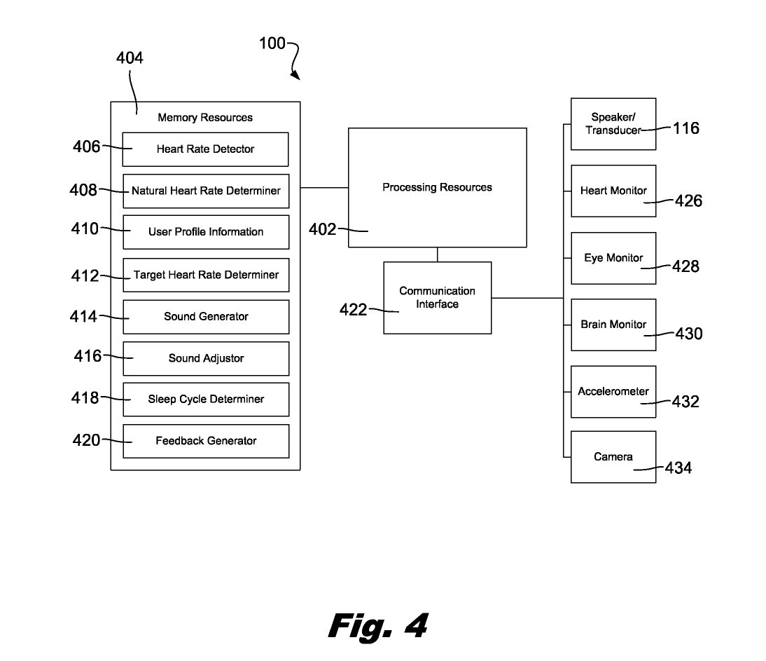

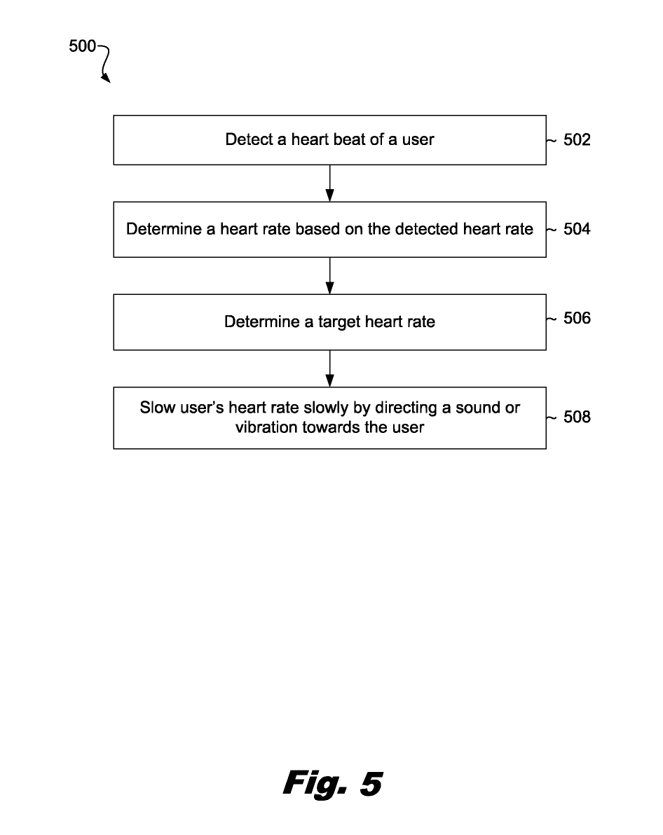

FIG. 4 illustrates a perspective view of an example of an adjustment system 100 in accordance with the present disclosure. The adjustment system 100 may include a combination of hardware and programmed instructions for executing the functions of the adjustment system 100. In this example, the adjustment system 100 includes processing resources 402 that are in communication with memory resources 404. Processing resources 402 include at least one processor and other resources used to process the programmed instructions. The memory resources 404 represent generally any memory capable of storing data such as programmed instructions or data structures used by the adjustment system 100. The programmed instructions and data structures shown stored in the memory resources 404 include a heart rate detector 406, a natural heart rate determiner 408, user profile information 410, a target heart rate determiner 412, a sound generator 414, a sound adjustor 416, a sleep cycle determiner 418, and a feedback generator 420.

The processing resources 402 may be in communication with communications interface 422 that communicates with external devices. Such external devices may include a speaker/transducer 116, a heart rate monitor 426, an eye monitor 428, a brain monitor 430, an accelerometer 432, a camera 434, another external device, or combinations thereof. In some examples, the processing resources 402 communicate with the external devices through a mobile device which relays communications between the processing resources 402 and the remote devices.

The external devices may gather information or execute a task to carry out a purpose of the adjustment system 100. For example, a speaker/transducer 116 may direct the sounds or vibrations towards the user in response to receiving a command from the processing resources 402. Further, the heart rate monitor 426 may collect information about the user's natural heart rate or at least the user's current heart rate, which can be used by the processing resources to determine which sounds to direct towards the user. The eye monitor 428 may be used to detect eye movement to assist the adjustment system 100 in determining whether the user is currently experiencing non-REM sleep or REM sleep.

The brain monitor 430 may be an electroencephalogram, a magnetoencephalogram, another type of brain monitor, or combinations thereof that can pick up waveforms generated by brain activity. As neurons in the brain fire, they create electrical signals that can be detected. During different stages of sleep, the brain's activity produces different types of patterns. For example, alpha waves usually have a frequency of 8.0 to 15.0 and are often exhibited during the first stage of non-REM and during REM sleep. Theta waves often exhibit a frequency of 4.0 to 7.0 hertz and are often exhibited during the second stage of non-REM sleep and REM sleep. A delta wave usually has a frequency of 1.0 to 4.0 hertz and is often exhibited during a third stage of sleep. During REM sleep, the user's brain activity often appears to be similar to when the user is awake. Thus, the brain monitor 430 may be used to determine the sleep cycle that the user is currently experiencing. As a result, the adjustment system 100 may tailor the target heart rate to be appropriate to the particular sleep stage being experienced by the user.

The accelerometer 432 may be used to determine whether the user is moving in his or her sleep. Such information may assist the adjustment system 100 in determining whether the user is in a deep sleep, REM sleep, an initial cycle of sleep, and so forth. Such information can be used to determine the appropriate target heart rate for the user based in part on the user's current sleep stage. A camera 434 may also be used to determine the user's body motions and/or restlessness.

Further, the communication interface may be in communication with a database that contains information about the user. An example of a database that may be compatible with the principles described herein includes the iFit program as described above. In some examples, the user information accessible through the communication interface includes the user's age, gender, body composition, height, weight, health conditions, other types of information, or combinations thereof that may be helpful in determining the appropriate target heart rate for the user.

The processing resources 402, memory resources 404 and external devices may communicate over any appropriate network and/or protocol through the communications interface 422. In some examples, the communications interface 422 includes a transceiver for wired and/or wireless communications. For example, these devices may be capable of communicating using the ZigBee protocol, Z-Wave protocol, BlueTooth protocol, Wi-Fi protocol, Global System for Mobile Communications (GSM) standard, another standard or combinations thereof. In other examples, the user can directly input some information into the adjustment system 100 through a digital input/output mechanism, a mechanical input/output mechanism, another type of mechanism or combinations thereof.

The memory resources 404 include a computer readable storage medium that contains computer readable program code to cause tasks to be executed by the processing resources 402. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory or combinations thereof.

The heart rate detector 406 represents programmed instructions that, when executed, cause the processing resources 402 to detect the heart rate of the user. This may be accomplished in response to the heart rate monitor 426 sending information to the processing resources 402. The natural heart rate determiner 408 represents programmed instructions that, when executed, cause the processing resources 402 to determine the natural heart rate of the user. Such a determination may be based on the information from the heart rate monitor 426.

The user profile information 410 may be stored in the memory resources 404 or in a database in communication with the processing resources 402 through the communications interface 422. Such user information may include data about the user's age, gender, health conditions, weight, body compositions, and so forth that may be used to determine the target heart rate for the user.

The target heart rate determiner 412 represents programmed instructions that, when executed, cause the processing resources 402 to determine the target heart rate. In some examples, known target heart rates that can be used for a wide variety of people to assist them with sleeping are used. In such an example, little personal data, if any, may be necessary to assist the user with sleeping. In other examples, the target heart rate is determined based on just the natural heart rate of the user. In such examples, the target heart rate determiner 412 may use an equation to determine the target heart rate. In some cases, the equation may be a percentage of the user's resting heart rate. For example, if the user is resting on the bed 102 and the natural resting heart rate of the user is 75 beats per minute, and the equation is

0.9 (resting heart rate)=target heart rate,

than the target heart rate may be determined to be 67.5 beats per minute. While this example has been described with a specific equation, any equation, procedure, or other mechanism for determining the user's target heart rate may be used in accordance with the principles described above.

The sound generator 414 represents programmed instructions that, when executed, cause the processing resources 402 to generate a sound or vibration that has a beat rate that is at least substantially similar to the target heart rate or at least a predetermined incremental beat customized to assist the user's heart rate to slowly adjust to the target heart rate. For example, the sound generator may cause a first sound to be directed towards the user that is slower than the user's current heart rate, but faster than the target heart rate.

The sound adjustor 416 represents programmed instructions that, when executed, cause the processing resources 402 to adjust the sounds as appropriate. For instance, if the sound directed to the user does not represent the target heart rate, the sound adjustor 416 causes the sound to be adjusted such that the sounds progressively get closer to the target heart rate. Likewise, as the user progresses through the sleep stages and/or sleep cycles, the target heart rate may change, and the sound adjustor 416 may cause sounds to change accordingly.

In some cases, the sounds generated by the sound generator 414 approximate a heartbeat. In other examples, the sounds are of different sounds, not a heartbeat, but include a beat that can be mimicked by the user's heart. For example, the sounds may be nature sounds, ocean sounds, bird sounds, animal sounds, music sounds, other types of sounds, or combinations thereof.

The sleep cycle determiner 418 represents programmed instructions that, when executed, cause the processing resources 402 to determine the sleep stage and/or sleep cycle of the user. This information may be used by the target heart rate determiner 412 to determine an appropriate target heart rate.

The feedback generator 420 represents programmed instructions that, when executed, cause the processing resources 402 to generate feedback to determine the effectiveness of the target heart rate. For example, if the sounds generated by the adjustment system 100 cause the user to fall asleep quickly, the feedback generator 420 may determine that the generated sounds were effective. However, if the sounds cause the user to have delayed sleep, to wake up, or to take longer than desired to fall asleep, the feedback generator may adjust the target heart rate and/or the intermediary sounds used to help the user's heart rate arrive at the target heart rate. In some examples, the beats of the intermediary sounds may be adjusted. In other examples, the increment times where the intermediary sounds are produced may be adjusted by the feedback generator to increase the effectiveness of the adjustment system 100. Thus, the adjustment system 100 may include one or more learning algorithms for increasing the effectiveness of helping the user to sleep.

Further, the memory resources 404 may be part of an installation package. In response to installing the installation package, the programmed instructions of the memory resources 404 may be downloaded from the installation package's source, such as a portable medium, a server, a remote network location, another location or combinations thereof. Portable memory media that are compatible with the principles described herein include DVDs, CDs, flash memory, portable disks, magnetic disks, optical disks, other forms of portable memory or combinations thereof. In other examples, the program instructions are already installed. Here, the memory resources 404 can include integrated memory such as a hard drive, a solid state hard drive, or the like.

In some examples, the processing resources 402 and the memory resources 404 are located within the heart rate monitor 426, the speaker 116, the bed 102, a component of the bed 102, the user's clothing, a mobile device, an external device, another type of device, or combinations thereof. The memory resources 404 may be part of any of these device's main memory, caches, registers, non-volatile memory, or elsewhere in their memory hierarchy. Alternatively, the memory resources 404 may be in communication with the processing resources 402 over a network. Further, data structures, such as libraries or databases containing user and/or workout information, may be accessed from a remote location over a network connection while the programmed instructions are located locally. Thus, the adjustment system 100 may be implemented with the mobile device, an external device, a phone, an electronic tablet, a wearable computing device, a head mounted device, a server, a collection of servers, a networked device, a watch, or combinations thereof. Such an implementation may occur through input/output mechanisms, such as push buttons, touch screen buttons, speech commands, dials, levers, other types of input/output mechanisms, or combinations thereof. Any appropriate type of wearable device may include, but are not limited to glasses, arm bands, leg bands, torso bands, head bands, chest straps, wrist watches, belts, earrings, nose rings, other types of rings, necklaces, garment integrated devices, other types of devices, or combinations thereof.

FIG. 5 illustrates a block diagram of an example of a method 500 for adjusting a natural heart rate in accordance with the present disclosure. In this example, the method 500 includes detecting 502 a heartbeat of a user, determining 504 a heart rate based on the detected heart rate, determining 506 a target heart rate, and slowing 508 the user's heart rate incrementally by directing a sound or vibration towards the user.

At block 502, the heart beat is detected. Such a heartbeat may be detected by the heart rate monitor or another type of device. At block 504, the natural heart rate is determined based at least in part on the detected heartbeat. In some examples, the heart rate is determined by counting the number of beats detected within a predetermined time period. In other examples, the signals from the heart rate monitor are filtered to remove noise or other distortions in the signal.

At block 506, the target heart rate is determined. The target heart rate may be based on applying an equation to the user's heart rate. In other examples, personal information about the user is also used to determine the target heart rate. For example, the user's age, gender, health, weight, body composition, historical sleeping heart rates and so forth may be used to determine the target heart rate.

At block 508, the user's heart is slowed by directing a sound or vibration toward the user. Such a sound or vibration may have a beat that is at least similar to the target heart rate. In other examples, the sound has a beat that is slower than the user's current heart rate, but faster than the target heart rate. The user's heart rate may be slowed in incremental stages or all at once.

### INDUSTRIAL APPLICABILITY

In general, the invention disclosed herein may provide the user with system for assisting the user with sleeping. Such a system may determine the user's natural or current heart rate with a heart rate monitor. The system may know or otherwise calculate a target heart rate to assist the user with sleeping. Such a target heart rate may be used to assist the user with falling asleep or staying asleep. Sounds that slow the user's heart to arrive at the target heart rate may be directed to the user. Such sounds may have a beat that is at least similar to the target heart rate. In other examples, the sounds are directed at slowly causing the user's heart rate to slow down to the target heart rate by using incremental beat rates in sounds directed at the user for specific periods of time. The incremental beat rates may progressively slow to the target heart rate.

The components of the adjustment system, such as a speaker and the heart rate monitor may be incorporated into a bed. In some examples, the speaker and/or the heart rate monitor are independent of the bed, but are in communication with the appropriate components of the adjustment system.

Such a system may be well suited for individuals who have sleeping disorders, especially those types of sleeping disorders that make it difficult for the user to relax when trying to fall asleep. However, the adjustment system, as described in the present disclosure, can also be used to help the user stay asleep. For example, by keeping the sounds directed at the user, the user's heart rate may stay at a desirable rate for sleeping. Further, the invention as described herein may be used to help the user progress through the various sleep stages and/or sleep cycles. For example, the heart rate may be increased to help the user progress from non-REM sleep to REM sleep. Likewise, the heart rate may be adjusted to help the user move from REM sleep to non-REM sleep to help the user wake up. For example, if the adjustment system determines that the user is in REM sleep just before the user's alarm is to go off, the adjustment system may cause a sound to be directed to the user to cause the user to transition from REM sleep to non-REM sleep. Such a system may assist the user in waking up without feeling groggy.

While the examples above have been described with reference to an adjustment system that assists a single person with sleeping, the principles described herein may be applied to assisting multiple users sleep at once. For example, the system may include multiple users where each user is associated with a dedicated heart rate monitor. In some cases, a single speaker may be used to direct sounds to both users simultaneously. In such an example, the sound may have a single beat that is customized to assist both users to fall asleep and/or stay asleep. In other examples, independent speakers or transducers may direct focused sounds or vibrations to each user such that the sounds affect the intended user without substantially affecting the unintended user. Such systems, with the dual speakers or just the single speakers may be incorporated into a double bed, a queen sized bed, a king sized bed, a twin sized bed, a hammock, a fold out bed, another type of bed, or combinations thereof.

What is claimed is:

1. A system for adjusting a heart rate of a user, comprising:
a heart rate monitor;
a transducer;
an interface for communicating with the heart rate monitor and the transducer; and
a processor and memory, the memory comprising programmed instructions to cause the processor to:
determine a natural heart rate of the user;
determine a target heart rate for the user; and
cause a vibration to be emitted from the transducer to adjust the natural heart rate to the target heart rate.

2. The system of claim 1, wherein the transducer comprises a speaker in communication with the interface.

3. The system of claim 2, wherein the vibration comprises a sound.

4. The system of claim 1, wherein the target heart rate is configured to assist the user sleep.

5. The system of claim 1, wherein the vibration is adjusted over a time period.

6. The system of claim 5, wherein the vibration at a beginning of the time period is closer to the natural heart rate and the vibration at an end of the time period is closer to the target heart rate.

7. The system of claim 5, wherein the vibration is changed incrementally during the time period.

8. The system of claim 1, wherein the heart rate monitor is incorporated into a bed.

9. The system of claim 1, wherein the transducer is incorporated into a bed.

10. The system of claim 9, wherein the vibration is propagated through a medium of the bed.

11. The system of claim 1, wherein the heart rate monitor is in wireless communication with the processor.

12. The system of claim 1, wherein the target heart rate is determined based on a percentage reduction of a resting heart rate of the user.

13. A system for adjusting a heart rate, comprising:
a communication interface;
a heart monitor in communication with the communication interface;
a speaker in communication with the communication interface; and
a processor and memory, the memory comprising programmed instructions to cause the processor to:
determine a natural heart rate of a user;
determine a target heart rate configured to assist the user with sleeping; and
cause a sound to be emitted from the speaker to adjust the natural heart rate to the target heart rate where the sound is adjusted over a time period such that the sound at a beginning of the time period is closer to the natural heart rate and the sound at an end of the time period is closer to the target heart rate.

14. The system of claim 13, wherein the sound is changed incrementally during the time period.

15. The system of claim 13, wherein the heart monitor is incorporated into a bed.

16. The system of claim 13, wherein the speaker is incorporated into a bed.

17. The system of claim 16, wherein the sound is propagated through a medium of the bed.

18. The system of claim 13, wherein the heart monitor is in wireless communication with the processor.

19. The system of claim 13, wherein the target heart rate is determined based on a percentage reduction of a resting heart rate of the user.

20. A system for adjusting a heart rate, comprising:
a communication interface;
a heart monitor incorporated into a bed and in communication with the communication interface;
a transducer incorporated into the bed and in communication with the communication interface; and
a processor and memory, the memory comprising programmed instructions to cause the processor to:
determine a natural heart rate of a user;
determine a target heart rate configured to assist the user with sleeping; and
cause a vibration to be emitted from the speaker to adjust the natural heart rate to the target heart rate where the vibration is adjusted incrementally over a time period such that the vibration at a beginning of the time period is closer to the natural heart rate and the vibration at an end of the time period is closer to the target heart rate.
